(19) United States
(12) Patent Application Publication
(10) Pub. No.: US 2024/0109856 A1
(43) Pub. Date: Apr. 4, 2024

(54) INTEGRATED FURFURAL OXIDATION AND FUROATE CARBOXYLATION PROCESS

(71) Applicant: UOP LLC, Des Plaines, IL (US)

(72) Inventors: Kristen E. Allaire, Chicago, IL (US); Veronica G. Deak, Chicago, IL (US); Yili Shi, Buffalo Grove, IL (US); Joel T. Walenga, Lake Zurich, IL (US)

(21) Appl. No.: 18/469,102

(22) Filed: Sep. 18, 2023

Related U.S. Application Data
(60) Provisional application No. 63/377,785, filed on Sep. 30, 2022.

(51) Int. Cl. C07D 307/68

(52) U.S. Cl. CPC C07D 307/68

(57) ABSTRACT

An integrated process for making furan-2,5-dicarboxylic acid (FDCA) and FDCA derivatives combines a furfural oxidation reaction, an intermediate water removal step, and a furoate salt carboxylation reaction. A furfural feed stream is oxidized with an alkaline solution in the presence of a catalyst and excess base with air as the oxidant. The resulting furoate solution from the first process step is dried to remove water. The anhydrous solid products are sent to a carboxylation process to produce FDCA salt, which can optionally be further reacted to form FDCA or FDCA ester.

### RELATED APPLICATIONS

This application claims priority to U.S. Provisional Patent Application Ser. No. 63/377,785, filed on Sep. 30, 2022, the entirety of which is incorporated herein by reference.

### BACKGROUND

Furan-2, 5-dicarboxylate (FDCA) and its derivatives may be used as a starting material for a family of biobased plastics as a replacement for such starting materials as terephthalic acid and p-xylene.

A current process for producing FDCA converts fructose to FDCA through an intermediate, hydroxymethylfurfural (HMF). This process has a number of disadvantages that lead to an expensive product, including the fact that fructose is an expensive starting material and that HMF has stability issues.

New processes for making FDCA would be desirable.

### BRIEF DESCRIPTION OF THE DRAWINGS

FIG. 1 is an illustration of one embodiment of an integrated furfural oxidation and furoate carboxylation process.

### DETAILED DESCRIPTION

An integrated process for making furan-2,5-dicarboxylic acid (FDCA) and FDCA derivatives has been developed. It combines a furfural oxidation reaction, an intermediate water removal step, and a furoate salt carboxylation reaction.

The oxidation process involves oxidizing a furfural feed stream with an alkaline solution in the presence of a catalyst and excess base with air as the oxidant. The resulting furoate solution from the first process step is dried to remove water. The anhydrous solid products are sent to a carboxylation process to produce FDCA salt.

The FDCA salt can be further reacted to form FDCA or FDCA derivatives such as esters.

Optionally, promoters and reagents for carboxylation reaction may be added prior to the water removal step.

The first step is the oxidation of a feed stream comprising furfural. The furfural and an aqueous alkali base stream (and optionally recycled carboxylates from downstream in the process) react in an oxidation reactor in the presence of a catalyst and an oxygen-containing gas to form a furoate salt in solution.

The feed stream typically comprises greater than 0 to 20 wt % furfural. In some embodiments, the furfural can be produced from pentoses obtained from biomass, making the process environmentally friendly.

The aqueous alkali base stream typically comprises greater than 0 to 20 wt % alkali base. Any alkali base can be used. Suitable alkali bases include, but are not limited to, Li, Na, K, and Cs.

Any catalyst suitable for the oxidation of furfural may be used. Suitable catalysts include, but are not limited to, copper, silver, gold, or combinations thereof. The catalyst may be supported on an inert carbonaceous or ceramic material. In some embodiments, the catalyst comprises a metal oxide. In some embodiments, the catalyst comprises silver.

The oxygen-containing gas typically comprises greater than 0 to 21% oxygen. Typically, air is used.

Typical reaction conditions for the oxidation reaction include a temperature in the range of 25 to 100° C., and a pressure in the range of 6.9 to 3447 kPa.

The reaction product contains furoate salt in residual unreacted alkali base, and unreacted carboxylates from the feed stream. There may be unreacted furfural as well as in the alkaline solution. The air is purged from the reactor.

The reaction mixture may also contain furfuryl alcohol, which is an undesirable byproduct of the oxidation reaction. The furfuryl alcohol may be removed using any suitable method, including, but not limited to, biphasic solvent extraction prior to the water removal step.

The reaction mixture is sent for liquid removal or crystallization. The feed is the solution of furoate salt and the alkaline solution. A promoter salt may optionally be included. Suitable promoter salts include, but are not limited to acetate and malonate salts.

The liquid (typically water (potentially mixed with a small amount of methanol)) is removed from the furoate salt in solution and alkaline solutions, leaving solid furoate salt and alkali salt, and optionally the promoter salt. Any suitable liquid removal process can be used. Suitable water removal processes include, but are not limited to drying and crystallization.

The solid furoate and alkali salts, and optionally the promoter salt are fed to the second reactor. In some embodiments, the furoate salt reacts with the CO₂-containing gas to form the FDCA salt in the second reactor. The alkali salt and any promoter salts present can be recycled to the oxidation reactor.

Suitable promoter salts for the reaction with the CO₂-containing gas include, but are not limited to, acetate and malonate salts. In some embodiments, the promoter salt comprises a carboxylate with at least one hydrogen on the carbon connected to the carboxylate.

Typical reaction conditions for the carboxylation reaction include a temperature in the range of 280 to 350° C., and a pressure in the range of 6.9 to 6900 kPa.

In other embodiments, the reaction in the second reactor is a disproportionation reaction (also known as a Henkel reaction). In this case, no CO₂ is used in the second reaction. Instead, the furoate salt is reacted with a carboxylate-containing compound. Optionally, a promoter may also be used in the reaction. Suitable promoters for the reaction with the carboxylate-containing compound include, but are not limited to, comprises a cadmium salt, a zinc salt, or a mercury salt. In some embodiments, the carboxylate-containing compound comprises an acetate. In some embodiments, the promoter is recycled.

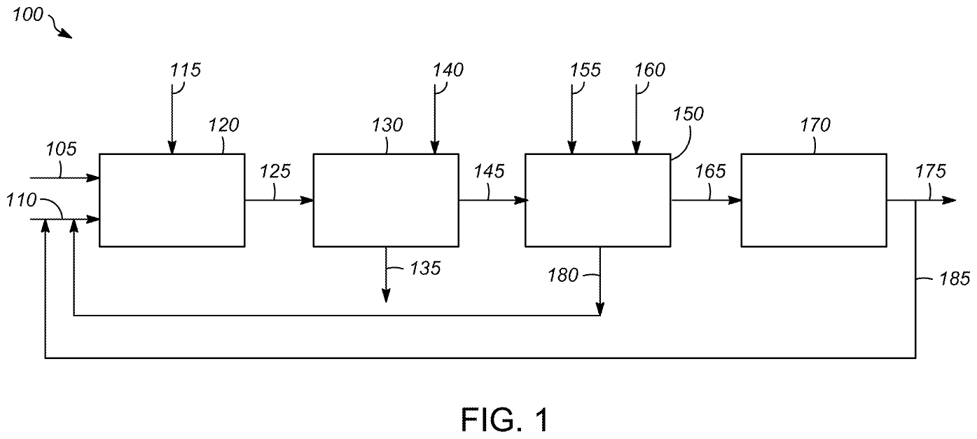

FIG. 1 illustrates one embodiment of the process 100 furan-2,5-dicarboxylic acid (FDCA) and FDCA derivatives. The feed stream 105 containing furfural, the aqueous alkali base stream 110, and an oxygen-containing gas stream 115 are sent to an oxidation reactor 120 containing the oxidation catalyst. The reaction produces a furoate salt in solution. The reaction mixture also contains the aqueous alkali base, unreacted furfural.

The effluent stream 125 is sent to a vessel 130 where water stream 135 is removed. The water can be removed by drying and/or crystallization. Other methods may also be used.

Optionally, a promoter salt 140 can be added prior to removing the water.

The water removal step results in a solid mixture of furoate salt and alkali salt. It will also contain the promoter salt, if it was added.

The stream 145 of solid mixture of furoate salt and alkali salt is sent to the second reactor 150. In some embodiments, the furoate salt is reacted with a CO₂-containing gas stream 155 to produce a FDCA salt. In other embodiments, the furoate salt is reacted with a carboxylate-containing compound 160 to produce an FDCA salt stream 165 containing the FDCA salt. In some embodiments, the FDCA salt can be recovered as a product.

In other embodiments, the FDCA salt stream 165 is sent to an acidification reactor 170 where the FDCA salt can be acidified by the addition of an acid to form FDCA. In another embodiment, the FDCA salt can be acidified and esterified. The product stream 175 from the acidification reactor 170 can be recovered.

In other embodiments, the FDCA salt stream 165 is sent to a carboxylate esterification reactor where the FDCA salt can be esterified with methanol and CO₂ to form FDME. The FDME product stream from the esterification reactor can be recovered. The un-recovered components from the FDME product stream can recycled to the initial oxidation reactor.

In other embodiments, the FDCA salt can be acidified and then esterified.

In some embodiments, a promoter recycle stream 180 from the second reactor 150 can be recycled to the oxidation reactor 120, to the effluent stream 125 from the oxidation reactor 120 and before the vessel 130.

In some embodiments, a portion 185 of the product stream 175 can be recycled to the oxidation reactor 120, to the effluent stream 125 from the oxidation reactor 120 and before the vessel 130.

### SPECIFIC EMBODIMENTS

While the following is described in conjunction with specific embodiments, it will be understood that this description is intended to illustrate and not limit the scope of the preceding description and the appended claims.

A first embodiment of the invention is a process for making furan-2,5-dicarboxylic acid (FDCA) and FDCA derivatives comprising oxidizing a feed stream comprising furfural and an aqueous alkali base stream in an oxidation reactor in the presence of a catalyst and an oxygen-containing gas to form a reaction mixture comprising a furoate salt in solution; removing liquids from the reaction mixture to form a solid comprising the furoate salt and an alkali salt; and reacting the furoate salt with a CO₂-containing gas, or a carboxylate-containing compound, or both in a second reactor to produce an FDCA salt. An embodiment of the invention is one, any or all of prior embodiments in this paragraph up through the first embodiment in this paragraph wherein reacting the furoate salt with the CO₂-containing gas or the carboxylate-containing compound comprises reacting the furoate salt with the CO₂-containing gas. An embodiment of the invention is one, any or all of prior embodiments in this paragraph up through the first embodiment in this paragraph wherein the feed further comprises a first promoter, wherein the reaction mixture further comprises a first promoter salt, wherein the solid further comprises the first promoter salt, and further comprising recycling the first promoter salt. An embodiment of the invention is one, any or all of prior embodiments in this paragraph up through the first embodiment in this paragraph wherein the first promoter salt comprises a carboxylate with at least one hydrogen on the carbon connected to the carboxylate. An embodiment of the invention is one, any or all of prior embodiments in this paragraph up through the first embodiment in this paragraph wherein reacting the furoate salt with the CO₂-containing gas, or the carboxylate-containing compound, or both comprises reacting the furoate salt with the carboxylate-containing compound. An embodiment of the invention is one, any or all of prior embodiments in this paragraph up through the first embodiment in this paragraph wherein the feed further comprises a second promoter, wherein the reaction mixture further comprises a second promoter salt, wherein the solid further comprises the second promoter salt, and further comprising recycling the second promoter salt. An embodiment of the invention is one, any or all of prior embodiments in this paragraph up through the first embodiment in this paragraph wherein the second promoter salt comprises a cadmium salt, a zinc salt, or a mercury salt. An embodiment of the invention is one, any or all of prior embodiments in this paragraph up through the first embodiment in this paragraph wherein the carboxylate-containing compound comprises an acetate. An embodiment of the invention is one, any or all of prior embodiments in this paragraph up through the first embodiment in this paragraph further comprising acidifying the FDCA salt to form the FDCA; or esterifying the FDCA salt to form an FDCA ester; or acidifying the FDCA salt to form the FDCA and esterifying the FDCA to form an FDCA ester. An embodiment of the invention is one, any or all of prior embodiments in this paragraph up through the first embodiment in this paragraph further comprising recycling a portion of the FDCA salt, the FDCA, or the FDCA ester to the oxidation reactor, the second reactor, or both. An embodiment of the invention is one, any or all of prior embodiments in this paragraph up through the first embodiment in this paragraph wherein the reaction mixture further comprises furfuryl alcohol, and further comprising removing the furfuryl alcohol from the reaction mixture before removing the liquid from the reaction mixture. An embodiment of the invention is one, any or all of prior embodiments in this paragraph up through the first embodiment in this paragraph further comprising recycling at least a portion of the alkali base, or at least a portion of the furoate or furoate salt, or at least a portion of a first promoter salt, or at least a portion of a second promoter salt, or combinations thereof to the oxidation reactor. An embodiment of the invention is one, any or all of prior embodiments in this paragraph up through the first embodiment in this paragraph wherein the catalyst comprises copper, silver, gold, or combinations thereof. An embodiment of the invention is one, any or all of prior embodiments in this paragraph up through the first embodiment in this paragraph wherein the catalyst is supported on either a carbonaceous or ceramic support. An embodiment of the invention is one, any or all of prior embodiments in this paragraph up through the first embodiment in this paragraph wherein the catalyst comprises a metal oxide. An embodiment of the invention is one, any or all of prior embodiments in this paragraph up through the first embodiment in this paragraph wherein removing water from the reaction mixture comprises heating the reaction mixture to evaporate the water or crystallizing the reaction mixture.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A process for making furan-2,5-dicarboxylic acid (FDCA) and FDCA derivatives comprising:
oxidizing a feed stream comprising furfural and an aqueous alkali base stream in an oxidation reactor in the presence of a catalyst and an oxygen-containing gas to form a reaction mixture comprising a furoate salt in solution;
removing liquids from the reaction mixture to form a solid comprising the furoate salt and an alkali salt;
reacting the furoate salt with a CO₂-containing gas, or a carboxylate-containing compound, or both in a second reactor to produce an FDCA salt.

2. The process of claim 1 wherein reacting the furoate salt with the CO₂-containing gas or the carboxylate-containing compound comprises reacting the furoate salt with the CO₂-containing gas.

3. The process of claim 2 wherein the feed further comprises a first promoter, wherein the reaction mixture further comprises a first promoter salt, wherein the solid further comprises the first promoter salt, and further comprising:
recycling the first promoter salt.

4. The process of claim 3 wherein the first promoter salt comprises a carboxylate with at least one hydrogen on the carbon connected to the carboxylate.

5. The process of claim 1 wherein reacting the furoate salt with the CO₂-containing gas, or the carboxylate-containing compound, or both comprises reacting the furoate salt with the carboxylate-containing compound.

6. The process of claim 5 wherein the feed further comprises a second promoter, wherein the reaction mixture further comprises a second promoter salt, wherein the solid further comprises the second promoter salt, and further comprising:
recycling the second promoter salt.

7. The process of claim 6 wherein the second promoter salt comprises a cadmium salt, a zinc salt, or a mercury salt.

8. The process of claim 5 wherein the carboxylate-containing compound comprises an acetate.

9. The process of claim 1 further comprising:
acidifying the FDCA salt to form the FDCA; or
esterifying the FDCA salt to form an FDCA ester; or
acidifying the FDCA salt to form the FDCA and esterifying the FDCA to form an FDCA ester.

10. The process of claim 9 further comprising:
recycling a portion of the FDCA salt, the FDCA, or the FDCA ester to the oxidation reactor, the second reactor, or both.

11. The process of claim 1 wherein the reaction mixture further comprises furfuryl alcohol, and further comprising:
removing the furfuryl alcohol from the reaction mixture before removing the liquid from the reaction mixture.

12. The process of claim 1 further comprising:
recycling at least a portion of the alkali base, or at least a portion of the furoate or furoate salt, or at least a portion of a first promoter salt, or at least a portion of a second promoter salt, or combinations thereof to the first reactor.

13. The process of claim 1 wherein the catalyst comprises copper, silver, gold, or combinations thereof.

14. The process of claim 13 wherein the catalyst is supported on either a carbonaceous or ceramic support.

15. The process of claim 1 wherein the catalyst comprises a metal oxide.

16. The process of claim 1 wherein removing water from the reaction mixture comprises heating the reaction mixture to evaporate the water or crystallizing the reaction mixture.
